(19) United States
(12) Patent Application Publication
(10) Pub. No.: US 2004/0186350 A1
(43) Pub. Date: Sep. 23, 2004

(54) Apparatus and methods for guiding an endoscope via a rigidizable wire guide

(75) Inventors: Rodney Brenneman, San Juan Capistrano, CA (US); Richard C. Ewers, Fullerton, CA (US); Vahid Saadat, Saratoga, CA (US); Eugene G. Chen, Carlsbad, CA (US)

Correspondence Address: TOWNSEND AND TOWNSEND AND CREW, LLP, TWO EMBARCADERO CENTER, EIGHTH FLOOR, SAN FRANCISCO, CA, 94111-3834, US

(73) Assignee: USGI Medical Corp., Saratoga, CA

(21) Appl. No.: 10/757,980

(22) Filed: Jan. 13, 2004

Related U.S. Application Data
(60) Provisional application No. 60/440,054, filed on Jan. 13, 2003.

(51) Int. Cl. A61B 1/00

(52) U.S. Cl. 600/146; 600/144

(57) ABSTRACT

The present invention provides methods and apparatus for guiding an endoscope during an endoluminal procedure via one or rigidizable wires. Ridgidizable, or shape-lockable, wires of the present invention have multiple states: they may be flexible to conform to various curvilinear paths, ridgidized to shape-lock in any path they obtain, and may be cycled between states. A rigidizable wire of the present invention advantageously may serve as a shape-lockable “backbone” within an endoscope, overtube, or endoscopic tool. In its flexible state, the wire can assume whatever shape the endoscope, overtube, or tool assumes as it is maneuvered. Once stiffened or rigidized, the wire forms the backbone to maintain the static shape of the endoscope, overtube, or tool. Methods of using apparatus of the present invention are also provided.

### CROSS-REFERENCES TO RELATED APPLICATIONS

This application claims benefit under 35 USC 119(e) of U.S. Provisional Patent Application Serial No. 60/440,054 (Attorney Docket No. 021496-000400US), filed Jan. 13, 2003, which is incorporated herein by reference in its entirety.

### BACKGROUND OF THE INVENTION

1. Field of the Invention.

The present invention relates to endoluminal procedures, endoscopic procedures and accessories for use with endoscopes. More particularly, the present invention relates to methods and apparatus for guiding an endoscope during an endoluminal procedure, such as trans-esophageal gastric treatment or colonoscopy, via a rigidizable wire guide.

2. Description of the Background Art.

Endoscopy is commonly used for visualization and treatment of a variety of diseases within the body. These include bronchoscopy, colonoscopy, thoracoscopy and laparoscopy. Passage of endoscopes through the esophagus for gastric and other upper gastro-intestinal (“G.I.”) procedures, as well as passage into the colon for lower G.I. procedures, is medically well known and widely practiced. However, both approaches face complications in traversing the tortuous paths of the upper and lower G.I. to reach the target site.

Colonoscopy involves passing a flexible endoscope through the anus and into the colon for diagnosis and treatment. Typical endoscopes are between about 12 and 19 mm in diameter and 130 to 190 cm in length. They include a fiberoptic bundle for image transmission and another bundle for transmitting light to illuminate the area of interest. The endoscope also includes at least one working channel for passing instruments needed for treatment, insufflation, irrigation or evacuation.

Lower G.I. endoscopy can typically reach as far as the cecum at the distal end of the colon. Achieving access to any portion of the colon involves a tortuous passage and tedious manipulation of the endoscope. Typically, as the scope passes around corners, further advancing tends to result in “looping-out”. This looping-out is characterized by a lack of movement of the distal tip while the middle portion of the endoscope tends to bow out in the direction of proximal scope advancement. It may also be difficult to maintain and manipulate the endoscope at a given location of treatment without some type of guide to maintain the column strength of the proximal endoscope within the patient.

Upper gastro-intestinal tract access involves use of an endoscope as described above, but passed through the esophagus to the stomach or upper regions of the small intestine. This involves traversing a considerable distance and possibly a difficult pathway to achieve access. If treatment is to be performed in the stomach, it is likely that the endoscope will need to be angled severely to achieve access. Treatment would then involve passing instruments through one or more of the working channels of the endoscope.

Various guide tubes and sheaths have been proposed for use over endoscopes. Applicant's co-pending U.S. patent application Ser. No. 10/281,462, filed Oct. 25, 2002, which is incorporated herein by reference in its entirety, describes a guide tube that can be rigidized to maintain the shape of the endoscope while it is advanced, using nested links and multiple wires to apply tension between links.

United States patent application publication 2002/0161281 to Jaffe et al. suggests using a guide tube that is passed over an endoscope and is comprised of segments with tensioning members that draw the segments together and lock the guide tube in whatever shape the endoscope has assumed.

U.S. Pat. No. 5,779,624 to Chang describes a simple overtube that straightens the sigmoid colon during endoscopy. Chang does not describe any stiffening or activation features.

U.S. Pat. No. 5,174,276 to Crockard describes a lockable steerable endoscope with tensioning members that can affect steering and cause adjacent conduit elements to bear and lock against each other. This device is also capable of delivering clips for occluding aneurysms.

United States patent application publication 2002/0120178 to Tartaglia et al. discloses a rigidizable tracking rod or guide that is placed within an endoscope working lumen and which allows the endoscope to slide over it in a mono-rail fashion. The guide, in its flexible state, is passed through the endoscope's working channel and then rigidized. The endoscope may then be further advanced over the rigidized guide.

U.S. Pat. No. 6,179,776 to Adams et al. describes a flexible sheath surrounding an endoscope and a pre-shaped wire that slides within a lumen of the sheath. The wire has a deflected natural state that it assumes when the sheath is advanced beyond the distal end of the endoscope.

U.S. Pat. No. 5,337,733 to Bauerfiend et al. describes insertion means that can be made rigid via evacuation of an intermediary space between inner and outer walls. A colonoscope may be advanced through a lumen of the insertion means, such that the insertion means act as a rigidizable overtube.

U.S. Pat. No. 5,251,611 to Zehel et al. describes a pair of concentric conduits. At least one of the concentric conduits may be stiffenable to act as a guide for advancement of the other conduit during an exploratory procedure.

In view of the foregoing, it would be desirable to provide methods and apparatus for guiding an endoscope that reduce tedious manipulation of the endoscope.

It would be desirable to provide methods and apparatus that reduce looping-out of the endoscope during lower G.I. endoscopy.

It would be desirable to provide methods and apparatus for guiding an endoscope that maintain or enhance column strength of the proximal endoscope when disposed within a patient.

It also would be desirable to provide methods and apparatus that facilitate and/or maintain severe angles to achieve access during upper G.I. endoscopy.

### BRIEF SUMMARY OF THE INVENTION

In view of the foregoing, it is an object of the present invention to provide methods and apparatus for guiding an endoscope that reduce tedious manipulation of the endoscope.

It is another object of the present invention to provide methods and apparatus that reduce looping-out of the endoscope during lower G.I. endoscopy.

It is yet another object to provide methods and apparatus for guiding an endoscope that maintain or enhance column strength of the proximal endoscope when disposed within a patient.

It is an object of the present invention to provide methods and apparatus that facilitate and/or maintain severe angles to achieve access during upper G.I. endoscopy.

These and other objects of the present invention are accomplished by providing apparatus comprising a rigidizable wire. The rigidizable, or shape-lockable, wire has multiple states: it can be flexible to conform to various curvilinear paths, rigidized to shape-lock in any path it obtains, and can be cycled between states.

A rigidizable wire of the present invention advantageously may serve as a shape-lockable “backbone” within an endoscope, overtube, or endoscopic tool. In its flexible state, it can assume whatever shape the endoscope, overtube, or tool assumes as it is maneuvered. Once stiffened or rigidized, the wire forms the backbone to maintain or “remember” the static shape of the endoscope, overtube, or tool.

In a preferred embodiment, the wire comprises a series of discrete segments that pivot in three dimensions relative to each other. The segments are strung together on a flexible cable that is attached to the proximal-most link and runs freely through all other links. In the flexible state the links have slack between each other and may re-orient relative to adjacent links. When tension is applied to the cable while the distal-most link or hub is held stationary, the slack is removed between the links. As the links engage each other, frictional forces develop that discourage further relative angulation. Various mechanisms for tensioning the wire are provided hereinafter. When so tensioned, the engaged links form a shape-locked wire guide.

Methods of using apparatus of the present invention also are provided.

### BRIEF DESCRIPTION OF THE DRAWINGS

The above and other objects and advantages of the present invention will be apparent upon consideration of the following detailed description, taken in conjunction with the accompanying drawings, in which like reference characters refer to like parts throughout, and in which:

FIGS. 1A and 1B are schematic views of apparatus in accordance with the present invention, shown in a flexible configuration and a shape-locked configuration, respectively;

FIGS. 2A-2C are schematic and sectional views of various exemplary nestable links from which the apparatus of FIG. 1 may be fabricated;

FIGS. 3A and 3B are side-sectional views of an illustrative embodiment of the apparatus of FIG. 1 fabricated from the nestable links of FIG. 2A, shown in the flexible configuration and the shape-locked configuration, respectively;

FIG. 4 is a side-sectional view of a first embodiment of a tensioning mechanism for use with apparatus of the present invention;

FIGS. 5A and 5B are side-views, partially in section, of alternative tensioning mechanisms for use with the present invention;

FIG. 6 is a side view, partially in section, illustrating a method of using apparatus of the present invention to shape-lock an overtube and guide an endoscope during an endoluminal procedure;

FIGS. 7A-7C are schematic views of illustrative embodiments of the overtube of FIG. 6;

FIG. 8 is a side view, partially in section, illustrating a method of using apparatus of the present invention to guide an endoscope during an endoluminal procedure via a rigidizable wire disposed within a working channel of the endoscope;

FIG. 9 is a schematic sectional view of a shape-lockable, split sheath overtube in accordance with the present invention;

FIGS. 10A-10C are schematic views illustrating a method of using the apparatus of FIG. 9 to facilitate colonoscopy;

FIGS. 11A and 11B are schematic views of apparatus of the present invention disposed within the working channel of an endoscope, illustrating a feature of the apparatus configured to reduce a risk of damaging the endoscope;

FIGS. 12A-12C are side-sectional detail views of illustrative embodiments of the nestable links of FIG. 2A;

FIGS. 13A-13C are schematic views, partially in section, illustrating a situation that may arise when guiding a conventional endoscope via a rigidizable wire guide inserted through the endoscope's working channel;

FIGS. 14A and 14B are a schematic view and a detail view, respectively, of an embodiment of the present invention configured to address the situation described with respect to FIG. 13; and

FIGS. 15A and 15B are schematic views illustrating a method of guiding a conventional endoscope with the apparatus of FIG. 14 disposed within the endoscope's working channel.

### DETAILED DESCRIPTION OF THE INVENTION

The present invention relates to endoluminal procedures, endoscopic procedures and accessories for use with endoscopes. More particularly, the present invention relates to methods and apparatus for guiding an endoscope during an endoluminal procedure, such as trans-esophageal gastric treatment or colonoscopy, via a rigidizable wire guide.

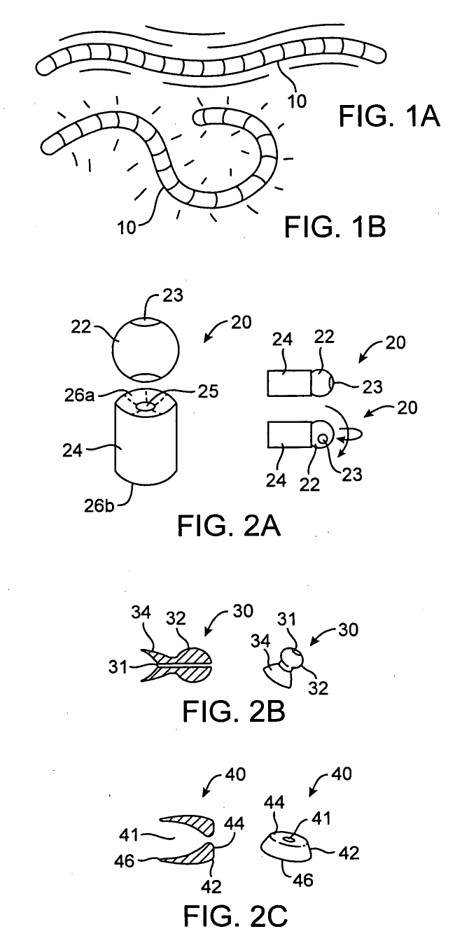

With reference to FIG. 1, apparatus of the present invention comprises rigidizable wire 10. Rigidizable, or shape-lockable, wire 10 has multiple states: it may be flexible to conform to various curvilinear paths, as seen in FIG. 1A, rigidized to shape-lock in any path it obtains, as in FIG. 1B, and may be cycled between the flexible and rigid states.

Wire 10 advantageously may serve as a shape-lockable “backbone” within an endoscope, overtube, or endoscopic tool. In the flexible state of FIG. 1A, it may assume whatever shape the endoscope, overtube, or tool assumes as it is maneuvered. Once transitioned to the stiffened or rigid state of FIG. 1B, the wire forms the backbone to maintain or “remember” the static shape of the endoscope, overtube, or tool.

Referring now to FIG. 2, wire 10 preferably is fabricated from a series of discrete segments that pivot in three dimensions relative to each other. The segments are strung together on a flexible cable that is attached to the proximal-most link and runs freely through all other links. In the flexible state, the links have slack between each other and may re-orient relative to adjacent links. When tension is applied to the cable while the distal-most link or hub is held stationary, the slack is removed between the links. As the links engage each other, frictional forces develop that discourage further relative angulations. When so tensioned, the engaged links transition wire 10 to the shape-locked configuration.

FIG. 2A illustrates a first exemplary embodiment of nestable links that may be used to fabricate wire 10. Nestable links 20 comprise spherical link 22 and cylindrical link 24. Cylindrical link 24 comprises end sockets 26a and 26b, in which spherical links 22 may be nested. Spherical link 22 comprises lumen 23, while cylindrical link 24 comprises lumen 25. The flexible cable may be passed through lumens 23 and 25, and a series or plurality of nestable links 20 may be strung together to form wire 10 from a column of pivoting links 22 and 24.

FIG. 2B illustrates alternative nestable links 30. Nestable links 30 comprise ball 32, socket 34 and lumen 31. Socket 34 is configured such that ball 32 of a first nestable link 30 may be nested within socket 34 of a second nestable link 30. A plurality of nestable links 30 may be stacked end-to-end to form wire 10 with a flexible cable disposed through lumen 31 and attached to the proximal-most link 30.

FIG. 2C illustrates alternative nestable links 40. Each link 40 comprises taper or radius 42, such that proximal end 44 is of smaller diameter than distal end 46. The proximal end of a first link 40 may be nested within the distal end of a second link 40. Links 40 further comprise lumen 41 through which the flexible cable may be disposed to form rigidizable wire 10. FIG. 2 have illustrated exemplary nestable links; additional nestable links will be apparent to those of skill in the art in view of this disclosure and are included within the present invention.

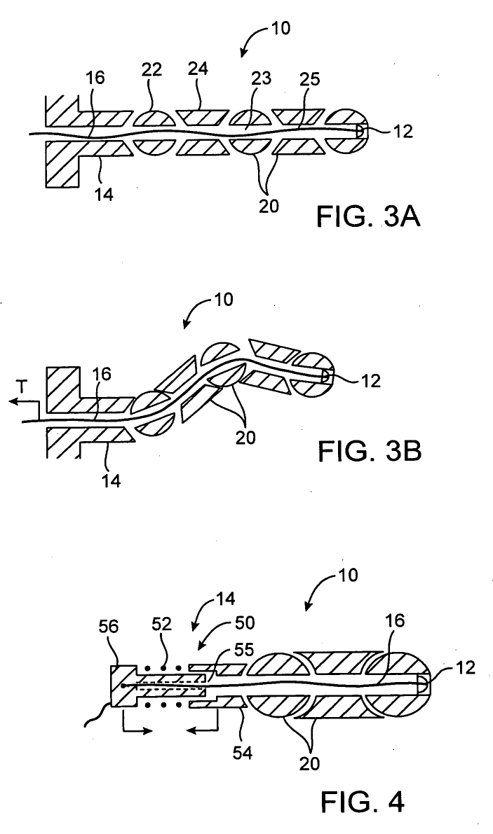

With reference now to FIG. 3, an embodiment of rigidizable wire 10 is described illustratively comprising a plurality of nestable links 20 of FIG. 2A. Wire 10 comprises proximal-most link 12; distal-most link, handle or hub 14; and flexible tensioning cable 16. Cable 16 is fixedly attached to proximal-most link 12 and passes freely through lumens 23 and 25 of spherical links 22 and cylindrical links 24, respectively, of nestable links 20. In FIG. 3A, no tension is applied to cable 16, and wire 10 therefore is disposed in the flexible configuration of FIG. 1A. In FIG. 3B, nestable links 20 have pivoted relative to one another, such that wire 10 comprises a tortuous geometry. Tension T has then been applied to wire 16. This results in application of a bearing load between adjoining links 20 of wire 10, which causes the wire to rigidize in the shape-locked configuration of FIG. 1B.

Distal link, handle or hub 14 preferably comprises a mechanism, such as a sprocket and pawl take-up spool, a linear take-up bar like that of a caulking gun, or a linear piston, to apply tension to cable 16. With reference to FIG. 4, an exemplary embodiment of such a mechanism is described. Hub 14 comprises spring-loaded hub tensioning mechanism 50. Mechanism 50 comprises compression spring 52 that is proximally coupled to female element 54 and is distally coupled to male element 56. Cable 16 passes through lumen 55 of female element 54 and distally terminates at male element 56.

Compression spring 52 dynamically separates male element 56 from female element 54, thereby tensioning cable 16 and disposing wire 10 in the rigidized, shape-locked configuration. Wire 10 may be transitioned to the flexible state by squeezing the male and female elements together to approximate the elements, thereby compressing spring 52 and releasing tension applied to cable 16. Wire 10 may be transitioned back to the rigidized state by releasing the male and female elements of mechanism 50, such that compression spring 52 once again separates the elements and tension is again applied to cable 16.

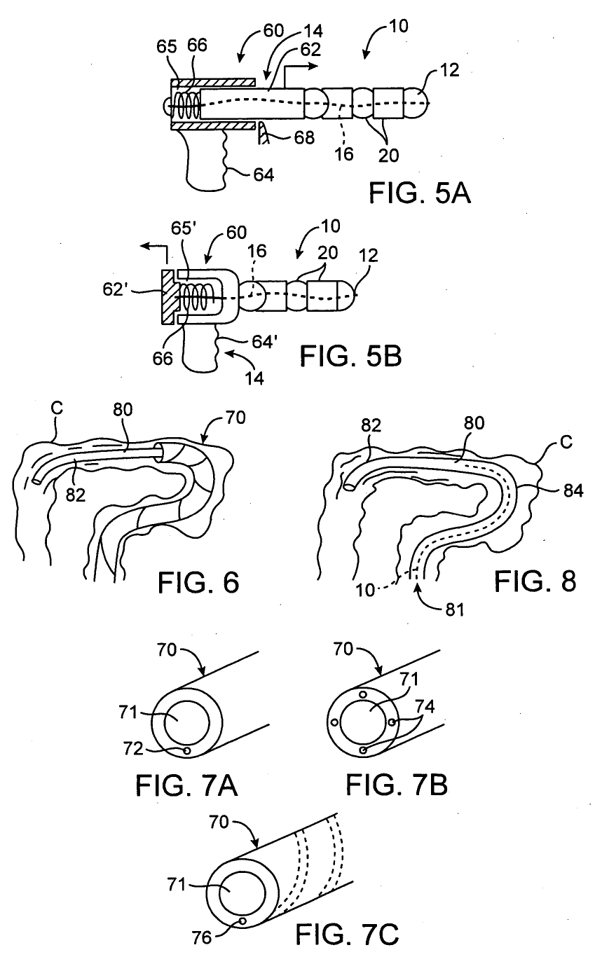

Mechanism 50 of FIG. 4 is configured such that, at rest, wire 10 is disposed in the shape-locked configuration. With reference to FIG. 5, alternative tensioning mechanisms are described, wherein wire 10 is disposed at rest in the flexible configuration. In FIG. 5A, tensioning mechanism 60 comprises tube 62, which acts as the distal-most link 14, handle 64 and tension spring 66. Handle 64 comprises chamber 65, in which spring 66 is disposed and coupled between the handle and tube 62. Furthermore, cable 16 is fixedly attached to handle 64 within chamber 65 (although alternative attachment points will be apparent).

In order to transition wire 10 from its at rest flexible configuration to the shape-locked configuration, handle 64 is held stationary while tube 62 is advanced, as illustrated by an arrow in FIG. 5A. Tube 62 is driven proximal along cable 16 to eliminate slack between links 20, thereby tensioning cable 16 and shape-locking wire 10. To return wire 10 to the flexible state, tube 62 is released, which causes tension spring 66 to dynamically approximate handle 64 and tube 62, thereby removing tension from cable 16.

FIG. 5A illustrates a “push forward” tensioning method. FIG. 5B illustrates a “pull back” method. In FIG. 5B, tensioning mechanism 60′ has been modified, such that handle 64′ acts as distal-most link 14, and tube 62 has been replaced with element 62′. Chamber 65′ of handle 64′ opens distally, and cable 16 is distally coupled to element 62′. In order to transition wire 10 to the shape-locked state, element 62′ is retracted distally relative to handle 64′, as illustrated by an arrow in FIG. 5B. Tension is applied to cable 16, and slack is removed from nested links 20. Wire 10 may be returned to the flexible state by releasing element 62′, which causes tension spring 66 to dynamically approximate element 62′ and handle 64′, thereby removing tension from cable 16.

FIG. 4 illustrates an embodiment of wire 10 that is at rest in the shape-locked configuration. FIG. 5 illustrate embodiments of wire 10 that are at rest in the flexible configuration. As will be apparent to those of skill in the art, embodiments of wire 10 alternatively may be disposed at rest in both the flexible and the rigid states, and the wire may be transitioned therebetween.

For example, tensioning mechanism 60 of FIG. 5A may comprise optional wedge 68 that may be advanced between tube 62 and handle 64 when tube 62 has been advanced proximal of handle 64. In this configuration, releasing tube 62 does not transition wire 10 back to the flexible configuration. Rather, wedge 68 precludes dynamic approximation of tube 62 and handle 64 via tension spring 66. Thus, wire 10 is at rest in the shape-locked configuration. Likewise, by removing wedge 68, wire 10 may be returned to an at rest flexible configuration.

Once stiffened, wire 10 forms a backbone that may be used to maintain or “remember” the static shape of an endoscope, overtube, or tool. With reference to FIG. 6, rigidizable wire 10 may, for example, be disposed as a locking spine within a lumen, e.g. a spiral lumen, formed in the sidewall of overtube 70. Wire 10 then may be selectively actuated to shape-lock overtube 70 in any desired configuration. Once the overtube is shape-locked, endoscope 80 may be advanced with or relative to overtube 70, for example, while the overtube is disposed within colon C. Shape-locked overtube 70 reduces tedious manipulation of endoscope 80, as well as a tendency for the endoscope to “loop-out”, since it forms a non-distensible surface for the scope to be pushed through.

With reference to FIG. 7, various techniques for housing wire 10 within overtube 70 are described. Overtube 70 comprises primary lumen 71 configured for advancement of endoscope 80 therethrough. As seen in FIG. 7A, locking spine wire 10 may be housed in solitary secondary longitudinal lumen 72 running the length of overtube 70. Alternatively, multiple locking spines 10 may be positioned in multiple longitudinal lumens 74 disposed in an annular pattern around overtube scope lumen 71, as in FIG. 7B. As a further alternative, in which the locking force is distributed uniformly over the overtube sidewall, a single helical locking spine 10 may be disposed in spiraled lumen 76 of the overtube, as in FIGS. 6 and 7C.

Referring now to FIG. 8, wire 10 alternatively may be advanced within working channel 81 of endoscope 80. Wire 10 may be advanced within the endoscope while disposed in a flexible configuration, then transitioned to a rigid configuration, as desired, to maintain an orientation of the endoscope. In this manner, wire 10 may reduce tedious manipulation of the endoscope, reduce looping-out, enhance column strength of the proximal endoscope and maintain severe angles traversed by the endoscope.

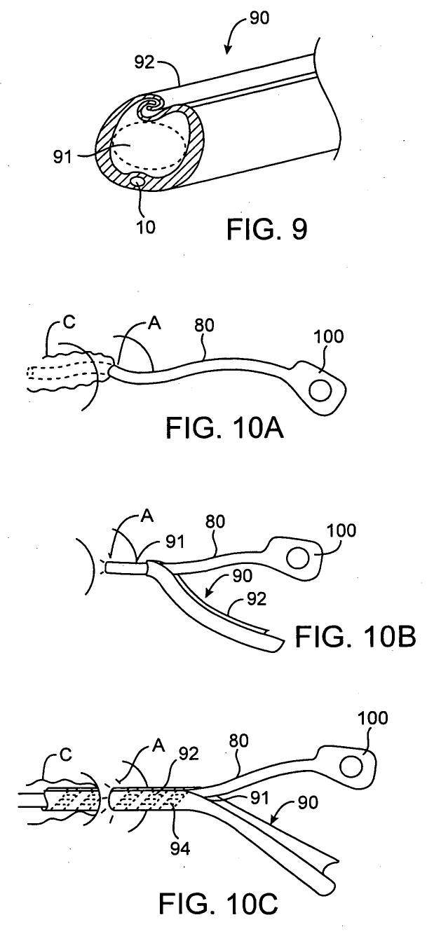

With reference to FIG. 9, in yet another embodiment, rigidizable wire 10 may act as a rigidizing spine within shape-lockable, split sheath overtube 90. Overtube 90 comprises lumen 91 configured for passage of endoscope 80 therethrough, as well as overlapping seam 92, which may be opened and closed selectively in a manner similar to a zipper or re-sealable bag. Seam 92 provides side access to lumen 91. Advantageously, the split sheath configuration of overtube 90 may be slid onto an endoscope that is already partially placed through, e.g., the colon or esophagus. In this manner, a medical practitioner may utilize the shape-lockable overtube selectively, for example, when the endoscope loops-out excessively or when further advancement of the endoscope presents a challenge.

With reference to FIG. 10, a method of using the apparatus of FIG. 9 during colonoscopy is described. In FIG. 10A, a routine colonoscopy has been initiated without the use of shape-lockable overtube 90. Endoscope 80 has been advanced through anus A into colon C. As the physician acquires depth of penetration, anatomical difficulties may arise that were not anticipated prior to the procedure. Accordingly, shape-lockable, split sheath overtube 90 may be removed from inventory and applied onto the scope via seam 92, as in FIG. 10B. Seam 92 may be closed about endoscope 80 external to the patient to form lumen 91 with the endoscope disposed therein. Overtube 90 then may be advanced to a depth that, upon shape-locking of the overtube via transition of wire 10 to the rigidized configuration, allows the clinician sufficient support to continue the procedure, as in FIG. 1C. Additional portions of seam 92 may be closed about endoscope 80, e.g., as overtube 90 is advanced. Seam 92 optionally may be more soundly secured in the closed configuration via secondary means, for example, surgical tape 94 wrapped around overtube 90 in a helical manner.

The split sheath configuration of overtube 90 allows the overtube to be used as needed and placed without losing scope positioning. It also has the benefit of consuming less exposed scope length outside of the anus. With this design, a physician can use the shape-locking overtube while maintaining fingertip access to the scope within a fraction of an inch of the anus, thus providing the physician a high degree of feel and control of the scope body.

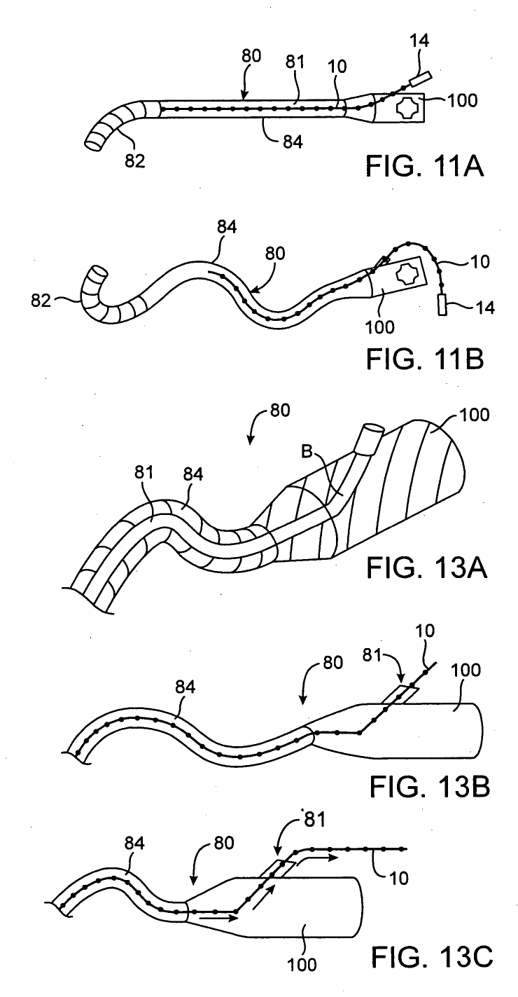

Referring now to FIG. 11, when rigidizable wire 10 forms a wire guide for endoscope 80, the endoscope may be advanced further while the distal endoscope section that rides over the wire is kept from “looping” out due to the non-distensible inner spine provided by wire 10—be it disposed within endoscope 80 or disposed within an overtube. Advantageously, unlike the system described in the Tartaglia publication, wire 10 of the present invention does not assume the shape of steerable tip 82 of endoscope 80. Rather, the present invention intentionally avoids allowing the rigidizable portion of wire 10 to even extend to steerable end 82 of the endoscope, so as to avoid a potential for damage to the endoscope if tip 82 were steered while wire 10 were rigidized therein.

As seen in FIG. 11A, wire 10 is dimensioned such that it cannot extend to steerable tip 82 of endoscope 80 when disposed within working channel 81 of the endoscope. Rather, wire 10 assumes the shape of non-steerable portion 84 of endoscope 80 and, when rigidized as in FIG. 11B, constrains movement of the endoscope as it slides over the rigidized wire. In use, wire 10 may be advanced within working channel 81 and then rigidized, for example, after endoscope 80 has been advanced within a patient's colon or esophagus. Alternatively, the wire may be disposed within the endoscope initially, i.e. prior to commencement of a medical procedure, and then rigidized as needed to facilitate further advancement of the endoscope.

As yet another alternative, wire 10 may be fixed within endoscope 80. Endoscope advancement then may be achieved, for example, via relative motion between the shape-lockable endoscope and an overtube. The overtube may be advanced while the endoscope is rigid, and the endoscope then may be advanced relative to the overtube while the endoscope is in the flexible configuration. In FIG. 11, wire 10 is disposed within working channel or lumen 81 of endoscope 80, but it should be understood that wire 10 alternatively may be disposed within a shape-lockable overtube, as described previously. The overtube would, for example, be dimensioned such that the shape-lockable portion of wire 10 could not extend to steerable end 82 of endoscope 80 when the overtube were advanced over the endoscope.

When rigidizable wire 10 of the present invention is utilized within the working channel of an endoscope, it preferably is used in conjunction with a multi-channel therapeutic endoscope or with an overtube that possesses alternate working channels. In use, the endoscope is worked into position with the wire 10 disposed in the flexible configuration, and then the wire is rigidized to maintain the shape of the endoscope at the target site or to facilitate further advancement of the endoscope. The wire is used to position and maintain the static position of the scope. However, the endoscope may be further advanced over the shape-locked wire. In this configuration, the shape-locked wire no longer guides the portion of the scope extending proximal beyond the wire; however, the distal section still benefits from the shape-locked guide and is kept from looping. Finally, the overtube with working channels is advanced over the endoscope, and instruments are placed therethrough for diagnosis or treatment; alternatively, additional channel(s) of the endoscope may be used for such advancement of instruments.

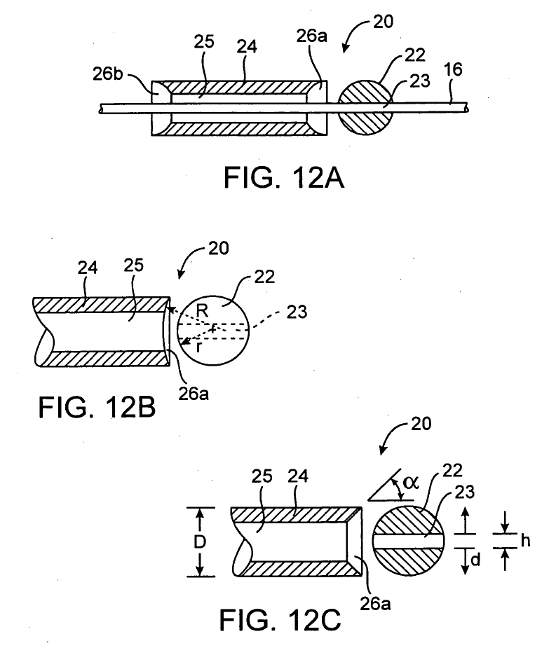

With reference now to FIG. 12, a more detailed description of nestable links 20, described previously with respect to FIG. 2A, is provided. As already discussed, nestable links 20 comprise spherical links 22 and cylindrical links 24. Each cylindrical link 24 comprises lumen 25 and end sockets 26a and 26b, in which spherical links 22 may be nested, and each spherical link 22 comprises lumen 23. Flexible cable 16 may be passed through lumens 23 and 25, as in FIG. 12A, and a series or plurality of nestable links 20 may be strung together to form wire 10 from a column of pivoting links 22 and 24. Cable 16 is permanently attached to a member at the proximal end of wire 10, while the distal end of the cable is attached to a tensioning element that is configured to pull on the cable while pushing on the distal-most nestable link 20. This creates high frictional forces at the interfaces of spherical links 22 and cylindrical links 24, which limit slipping between the links and rigidize/shape-lock wire 10.

The interfaces between the cylindrical and spherical links significantly influence a degree of rigidization that may be achieved with a given amount of cable tension. Fabricating the cylindrical links and/or the spherical links of nestable links 20 from relatively soft metals, polymers or composites may provide a degree of plastic compressibility that yields relatively high friction under tension. Interface contact angle also impacts rigidization efficiency.

For certain materials, a relatively large surface contact area results in higher friction. When utilizing such materials, the radius of curvature R of end sockets 26 of cylindrical links 24 preferably is substantially equal to the radius of curvature r of spherical links 22, as in FIG. 12A. For other materials, a point contact or minimal surface contact would result in the plastic deformation effect described above. For these materials, a configuration as shown in FIG. 12B may be utilized, wherein radius R is greater than radius r. Alternatively, a configuration as in FIG. 12C may be provided, wherein a straight chamfer of angle □ is used to finish end sockets 26. No chamfer may also be used to create this effect. As yet another alternative, radius R may be less than radius r, in order to create a wedging effect that may work best with some fabrication materials. As will be apparent, a degree of such wedging would have to be specified in order to avoid a locking action that would not release.

Another aspect of the present invention is the relative sizes, i.e. diameters d and D, respectively, of spherical links 22 and cylindrical links 24. It is desirable for the links to be of similar sizes, but not necessarily the same size. Assuming that links 22 and 24 are fabricated from the same material, fabricating rigidizable wire 10 from spherical links 22 of diameter d that are larger than the diameter D of cylindrical links 24 is expected to reduce sliding friction of wire 10 inside a lumen. This would occur because the total surface area of wire 10 in contact with a wall of the lumen would be reduced relative to the configuration wherein diameter d is equal to or less than diameter D. Greatest sliding friction would be expected when diameters D and d are equal. It is expected that providing spherical links 22 with a smaller diameter than that of cylindrical links 24 would yield the wedging effect described above.

Yet another aspect of the present device impacting the performance of wire 10 is a stiffness of cable 16, as well as a relative size or diameter of the cable as compared to the diameters of lumens 23 and 25 of spherical links 22 and cylindrical links 24, respectively (the diameter of lumens 23 is illustratively shown in FIG. 12C as h). This aspect directly influences the flexibility of rigidizable wire 10. It is desirable to have a greater diametric difference between cable 16 and lumens 25 of cylindrical links 24 than between the cable and lumens 23 of spherical links 22. This is because cylindrical links 24 preferably are longer than spherical links 22 and, thus, constrain bending of cable 16 more than the spherical links.

It is desirable to have a diametric difference between cable 16 and cylindrical links 24 of at least half of the cable diameter (at least a 50% cable diameter gap). More preferable is a diametric difference of at least one cable diameter (100% gap). It is desirable to have at least 25% of the cable diameter as a gap between the cable and spherical links 22. More preferably, a 30% to 50% cable diameter gap will be maintained. Chamfering the terminuses of lumens 23 of the spherical links may lessen this gap. Chamfering allows the cable to bend around a smaller arc without being constrained by the terminuses. These features also make it possible to rigidize the wire while it is disposed in a tighter bend radius.

In yet a further alternative embodiment of the rigidizable wire of the present invention, the spherical and cylindrical portions of nestable links 20 are combined by radiusing or chamfering the concaved end of the cylinder, while radiusing a convexed profile on the other end of the cylinder. This is similar to nestable links 40 of FIG. 2C. Preferably, a smaller radius is radiused on the concaved end than on the convexed end. Advantageously, this differential radius configuration is expected to provide the wedging action described earlier. Yet a further configuration comprises a chamfer on the concaved end and a radius on the convexed end. The advantage of such a link in a rigidizing wire is that it encourages plastic deformation of the links when made of softer materials or lower friction materials.

With reference now to FIG. 13, guiding a conventional endoscope via a rigidizable wire guide inserted through the endoscope's working channel, such as working channel 81 of endoscope 80, may prove challenging. As seen in FIG. 13A, endoscope 80 may terminate at rigid handle 100 that does not have a straight-through working channel. Rather, working channel 81 typically exits flexible portion 84 of endoscope 80 in an axial path, and then curves slightly out of axis to form bend B.

While disposed in its flexible state, rigidizable wire 10 of the present invention can easily pass through rigid handle 100, as seen in FIG. 13B. However, as seen in FIG. 13C, if wire 10 is rigidized and scope 80 is further advanced while the wire is held stationary, the portion of rigid wire 10 disposed within handle 100 will be forced to conform, contrary to its rigidity, to the tortuous geometry of working channel 81 through scope handle 100. This may result in damage to the wire, the endoscope or both, or may impede advancement of the endoscope relative to the wire.

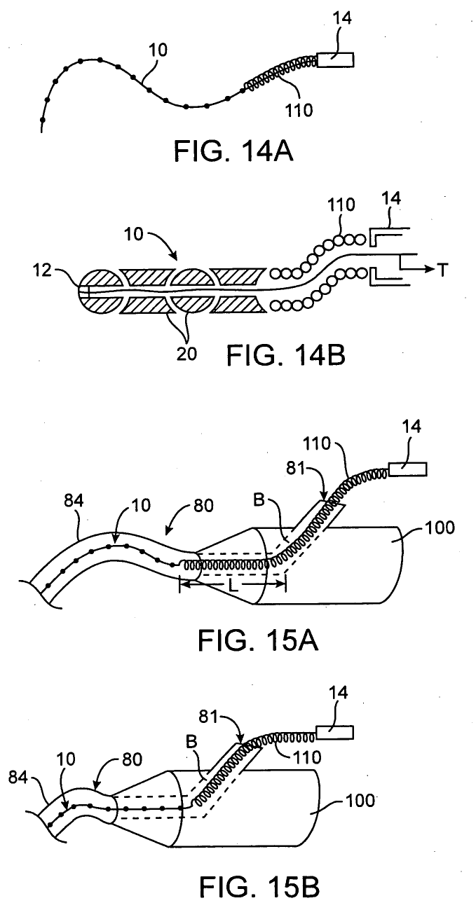

With reference to FIG. 14, an embodiment of wire 10 is described that is configured for advancement through the tortuous working channel of a conventional endoscope. FIG. 14A provides a schematic view of the apparatus, while FIG. 14B provides a detail view. In FIG. 14, the distal-most portion of wire 10 comprises tightly wound coil spring 110. The spring transmits tensile loads, yet still provides a degree of flexibility, such that wire 10 may be advanced within working channel 81 of endoscope 80 through rigid handle 100.

Referring to FIG. 15, when used in conjunction with conventional endoscope 80, wire 10 may be provided with a specified length L of coil spring 110, e.g. about 10 cm, that extends beyond bend B of working channel 81 within rigid handle 100 of endoscope 80, when wire 10 is fully inserted within the working channel. As seen in FIG. 15A, when fully inserted, wire 10 may be rigidized throughout a significant length of flexible portion 84 of endoscope 80 (and thereby a significant portion of the intubated anatomy). As seen in FIG. 15B, upon rigidization, coil spring 110 facilitates incremental advancement of scope 80 relative to rigidized wire 10 over a distance roughly equal to specified length L, i.e. advancement of scope 80 until the rigidized portion of wire 10 abuts bend B of working channel 81.

Further advancement of scope 80 along rigidized guide 10 may be achieved by transitioning wire 10 back to the flexible configuration, fully reinserting the wire within working lumen 81, transitioning the wire back to the rigid, shape-locked configuration, and re-advancing the endoscope relative to the rigid wire. Such reciprocating motion may be repeated, as necessary: the scope is advanced length L along rigid wire 10, the wire is relaxed, the wire is advanced length L and locked, the scope is again advanced length L, etc. As will be apparent to those of skill in the art, instead of, or in combination with, providing wire 10 with spring 110, scope 80 may be modified to comprise a straight working channel or semi-flexible or flexible channel through handle 100, thereby mitigating the difficulty encountered in passing wire 10 through the handle when the wire is disposed in the rigid configuration.

Although preferred illustrative embodiments of the present invention are described above, it will be evident to one skilled in the art that various changes and modifications may be made therein without departing from the invention. It is intended in the appended claims to cover all such changes and modifications that fall within the true spirit and scope of the invention.

What is claimed is:

1. Apparatus for intubation of a body cavity or lumen, the apparatus comprising:
an endoscope having a steerable tip; and
a wire guide,
wherein the apparatus is configured to preclude advancement of the wire guide to the steerable tip.

2. The apparatus of claim 1, wherein the wire guide is configured to reversibly transition between a flexible state and a substantially shape-locked state.

3. The apparatus of claim 2, wherein the wire guide is configured for advancement relative to a flexible body of the endoscope.

4. The apparatus of claim 2, wherein the wire guide is configured for advancement within a working channel of the endoscope.

5. The apparatus of claim 2, wherein the wire guide is disposed within an overtube, the overtube configured for advancement over the endoscope.

6. The apparatus of claim 1, wherein a length of the wire guide precludes advancement of the wire guide to the steerable tip.

7. The apparatus of claim 1, wherein the wire guide comprises a hub that precludes advancement of the wire guide to the steerable tip.

8. The apparatus of claim 4, wherein the wire guide comprises a section that remains flexible when the wire guide is disposed in the shape-locked state.

9. The apparatus of claim 5, wherein the overtube comprises a split sheath.

10. Apparatus for guiding an endoscope having a working channel with a substantially rigid tortuous section, the apparatus comprising:
a shape-lockable wire guide configured for advancement through the working channel,
wherein the wire guide is configured for relative advancement of the endoscope when the wire guide is shape-locked and disposed within the rigid tortuous section.

11. The apparatus of claim 10, wherein the wire guide comprises a flexible section that remains flexible when the wire guide is shape-locked.

12. The apparatus of claim 11, wherein the flexible section is configured for disposal within the rigid tortuous section of the working channel when the wire guide is shape-locked.

13. The apparatus of claim 11, wherein the flexible section comprises a coil spring.

14. The apparatus of claim 10, wherein the apparatus is configured to preclude advancement of the wire guide to a steerable tip of the endoscope.

15. Apparatus for guiding an endoscope, the apparatus comprising a shape-lockable overtube configured for advancement relative to the endoscope,
wherein the overtube comprises a seam for reversibly opening the overtube.

16. The apparatus of claim 15, wherein the overtube further comprises a rigidizable wire spine configured to reversibly shape-lock the overtube.

17. The apparatus of claim 15, wherein the seam facilitates reversibly disposing the shape-lockable overtube over the endoscope.

18. The apparatus of claim 16, wherein the apparatus is configured to preclude advancement of the rigidizable wire spine to a steerable tip of the endoscope.

19. A method for intubating a body cavity or lumen, the method comprising:
advancing an endoscope within the body cavity or lumen;
advancing a wire guide within the body cavity or lumen along the endoscope;
shape-locking the wire guide; and
advancing the endoscope along the shape-locked wire guide,
wherein a steerable tip of the endoscope always extends beyond the shape-locking wire guide.

20. The method of claim 19, wherein advancing the wire guide along the endoscope further comprises advancing the wire guide within a working channel of the endoscope.

21. The method of claim 19, wherein advancing the wire guide along the endoscope further comprises disposing the wire guide within an overtube, and advancing the overtube over the endoscope.

22. A method for intubating a body cavity or lumen, the method comprising:
advancing an endoscope within the body cavity or lumen;
advancing a split sheath overtube within the body cavity or lumen over the endoscope;
shape-locking the overtube; and
advancing the endoscope along the shape-locked split sheath overtube.
